(19) United States
(12) Patent Application Publication
(10) Pub. No.: US 2009/0035793 A1
(43) Pub. Date: Feb. 5, 2009

(54) Microchip for cell response evaluation

(75) Inventors: Taito Nishino, Tokyo (JP); Masaki Kitahara, Minamisaitama-gun (JP); Manabu Tokeshi, Kawasaki-City (JP); Takehiko Kitamori, Tokyo (JP)

Correspondence Address: OLIFF & BERRIDGE, PLC, P.O. BOX 320850, ALEXANDRIA, VA, 22320-4850, US

(73) Assignees: Nissan Chemical Industries, Ltd., Tokyo (JP); Kanagawa Academy of Science and Technology, Kawasaki City, Kanagawa (JP)

(21) Appl. No.: 11/988,731

(22) PCT Filed: Aug. 31, 2006
(86) PCT No.: PCT/JP2006/317715

§ 371 Date: Feb. 11, 2008

(30) Foreign Application Priority Data
Aug. 31, 2005 (JP) 2005-252084
Oct. 7, 2005 (JP) 2005-294727

(51) Int. Cl. G01N 33/53; C12M 1/00; C12Q 1/02

(52) U.S. Cl. 435/7.92; 435/287.1; 435/29

(57) ABSTRACT

In a microchip which enables cell cultivation and accurate cell count measurement, fine particles affixed with cells are trapped within a passage by making the minimum width of a solution and fine particle inlet into a cell culture portion larger than the maximum diameter of the fine particles, and making the width of an outlet smaller than the maximum diameter of the fine particles.

### TECHNICAL FIELD

The present invention relates to a microchip used in drug development, biotechnology research, and so on, and more particularly to a microchip used to cultivate cells and evaluate their functions and responses.

### BACKGROUND ART

In the fields of pharmaceutical drug development, pesticide development, food inspection, diagnosis, genetic engineering, and so on, evaluations using cells are widely performed. Conventionally, when evaluating the functions and responses of cells, the cells are cultivated in a culture medium or the like placed in a dish or similar object and used in various experiments. However, in cases where the response of the cells to a large number of chemicals and introduced substances is checked, there is a need for an evaluation method with which a specific cell reaction can be subjected to many types of analysis efficiently and within a short time period, and with which results can be obtained using a small amount of samples.

Under these conditions, many trials have taken place in recent years to observe and measure the chemical reactions of cells that are carried on a glass substrate or the like known as a microchip in the hope that the analysis period can be shortened by simplifying and automating the operation. A system using a microchip is considered advantageous because the amount of required samples and reagents and the amount of discharged waste liquid are reduced, and a portable system can be realized in a limited space and at a low cost. Research is underway into methods of applying these advantages in reality in order to evaluate cell functions and responses using a microchip in which the cells are adhered to the surface of a passage wall (see Analytical Chemistry 2005: 77, p. 2125-2131, Analytical Chemistry 2005: 77, p. 667-672, and Japanese Patent Application Publication No. JP-A-2003-294741, for example).

### DISCLOSURE OF THE INVENTION

However, in conventional cell cultivation using a microchip, the material of the substrate is not suitable for cell adhesion, and moreover, the effect of an extremely weak flow is great on an extremely small amount of solution, and hence the cells do not bond efficiently to the passage wall surface within the micropassage, making it difficult to hold a fixed number of cells within a fixed region of the passage. Methods exist for encouraging cell adhesion using means for chemically or physically modifying the inner surface of the passage, but in such cases, all of the passage wall surfaces are modified, and hence it becomes difficult to cultivate the cells only in target locations. Moreover, a microchip having an optimum passage surface for a particular type of cell must be manufactured for each cell type, and it is difficult to create minute microchip passages through the application of microprocessing using materials suited to cell adhesion. Further, when measuring the number of cells adhered to the passage before and after evaluating the functions and responses of the cells, the number of cells in a fixed region of the passage must be counted using a microscope in cases where the number of cells cannot be measured using a coloring reagent or the like, which is troublesome and time-consuming. Moreover, the surface area of the microchip passage is small, and hence it is difficult to increase the number of cells that can be adhered to a fixed region on the surface of the passage wall.

An object of the present invention is to provide a microchip with which cells can be held quantitatively in a specific location of a microchip passage using simple and efficient means such that cell cultivation and subsequent cell function evaluation can be performed in the specific location.

The present invention relates to:

(1) a microchip for evaluating a cell response, characterized in comprising a cell culture portion which is capable of holding a fine particle to which a cell is adhered;
(2) the microchip according to (1), characterized in comprising a passage formed in the interior of the microchip so as to be capable of carrying a liquid, a liquid inlet and outlet connected to two end portions of the passage, and a cell culture portion provided in the interior of the passage, which is capable of holding fine particles with a diameter of no more than 1 mm;
(3) the microchip according to (1) or (2), comprising a substance analysis portion provided in a latter half of the passage in relation to the cell culture portion for measuring a secretion from the cell;
(4) a microchip, wherein the passage outlet of the microchip according to (1) or (2) and a passage inlet of a microchip having a substance analysis portion are connected by an external tube;
(5) a method of measuring a cell secretion using the microchip according to (3) or (4);
(6) a method of measuring a cell secretion by performing an enzyme immunoassay in the substance analysis portion according to (3) or (4);
(7) the measuring method according to (5) or (6), wherein measurement is performed while causing a liquid to flow along the passage of the microchip continuously;
(8) the measuring method according to any one of (5) through (7), wherein the cell secretion is apolipoprotein A1;
(9) the measuring method according to any one of (5) through (7), wherein the cell secretion is apolipoprotein B;
(10) a screening method for a drug which is active in varying an amount of secretion from a cell, using the measuring method according to any one of (5) through (7);
(11) the drug screening method according to (10), wherein the cell secretion is apolipoprotein A1;
(12) the drug screening method according to (10), wherein the cell secretion is apolipoprotein B;
(13) the drug screening method according to (11), wherein the cell is a HepG2 cell; and
(14) the drug screening method according to (12), wherein the cell is a Caco-2 cell.

The microchip of the present invention specifies a microchip having a passage and other parts required for solution reactions and processes, which are formed in a substrate, and hence there are no particular limitations on the size of the microchip.

In the present invention, a cell culture portion is provided in a passage, and the width of the outlet thereof is made to be sufficiently smaller than the maximum diameter of fine particles to which cells can be adhered. As a result, the fine particles affixed with the cells can be prevented from flowing out of the cell culture portion when charged into the cell culture portion, and hence processing such as cell cultivation and drug addition can be performed in a fixed region of the microchip. Furthermore, a substance analysis portion is provided in the passage downstream of the cell culture portion, and therefore a substance secreted by the cells in the cell culture portion can be measured continuously.

There are no particular limitations on the method of forming the substance analysis portion as long as it is capable of detecting products produced by the cells in the cell culture portion selectively and with a high degree of sensitivity.

More specifically, the microchip of the present invention comprises a passage formed in the interior of a substrate so as to be capable of carrying a liquid and fine particles, a liquid inlet and outlet connected to the two end portions of the passage, a cell culture portion provided in the interior of the substrate at a midway point in the passage, which is capable of trapping fine particles to which cells are adhered, and a substance analysis portion connected to the cell culture portion by a single passage, all of which are formed integrally with the substrate. The present invention also includes a microchip in which the passage of a microchip comprising the cell culture portion and the passage of a different microchip comprising the substance analysis portion are connected by a tube.

The cell culture portion is set to have a larger volume than the passage along which the solution or fine particles flow, and is therefore capable of holding a large number of fine particles affixed with cells. Hence, in comparison with a case in which the cells are adhered to a passage wall surface, a larger number of cells can be held in the cell culture portion. Further, by measuring the number of cells adhered to the fine particles in advance, the number of fine particles introduced into the cell culture portion can be adjusted, and as a result, a fixed target number of cells can be held in a fixed region. At this time, a coloring method or a fluorescence method may be used to measure the number of cells on the fine particles, and therefore cell count measurement can be performed more easily than with a microscope observation method or the like. Further, the fine particles are charged into the interior of the cell culture portion, and hence the introduced solution creates turbulence and is agitated thereby. Thus, a drug sample or the like can be caused to act effectively on the cells held in the cell culture portion. Furthermore, the fine particles can be selected in advance from various materials which are suitable for cell adhesion, and as a result, cells can be held and cultivated, which would be impossible with a method of modifying the passage wall surface of the microchip.

Moreover, in the microchip of the present invention, the cell culture portion and substance analysis portion are connected by a single passage, and hence a substance secreted by the cells in the cell culture portion is carried directly to the substance analysis portion on the flow of liquid in the passage and trapped therein. As a result, the meritorious effects described below, which cannot be obtained from a device such as a microtiter plate employing static conditions with no liquid flow, can be exhibited.

1. A situation in which the substance secreted in the cell culture portion saturates the cell culture portion, leading to secretion inhibition due to feedback, can be avoided.
2. The secreted substance is not readsorbed or reabsorbed into the cells, and hence all of the substance secreted by the cells can be analyzed.
3. The substance secretion per unit time can be measured easily.
4. Analysis of the substance secreted by the cells in response to shearing stress produced by the liquid is easy.

Hence, when an apolipoprotein A1 (ApoA1 hereafter) receptor, for example, is expressed on the cell surface, the secretion of ApoA1 protein from HepG2 cells, which would be readsorbed into the cells under static conditions, can be measured easily.

Similarly, the amount of apolipoprotein B (ApoB hereafter) secreted from human colon cancer Caco-2 cells can also be measured easily.

Further, the secretion of a substance which exhibits transient expression and secretion in relation to a specific stimulation (for example, insulin secreted from β cells) or the like can also be measured easily.

Moreover, since the flow velocity of the passage can be varied easily, a substance which is secreted in response to shearing stress (for example, nitrogen monoxide secreted from vascular endothelial cells) or the like can also be measured easily.

The measurement described above cannot be achieved with a pre-existing device which employs static conditions with no liquid flow.

### BRIEF DESCRIPTION OF THE DRAWINGS

FIG. 1 is a transverse sectional view of a microchip according to the present invention; and

FIG. 2 is a top view of the microchip according to the present invention.

### BEST MODES FOR CARRYING OUT THE INVENTION

The present invention will be described in further detail below.

Examples of the cells that can be subjected to cell response evaluation using the microchip of the present invention include animal cells, plant cells, bacteria, mold, yeast, and viruses. Of these cells, animal cells are preferable when used in the screening of drugs such as medicines. Animal cells denote cells extracted from the living tissue of organs, tissue and so on within the body of an animal, including a human being, and there are no particular limitations thereon as long as the cells are obtained from living tissue. Examples of these cells include epithelial cells, endothelial cells, mesenchyme cells, or blood cells. Specific examples of epithelial cells include epidermal cells, hair papilla cells, corneal epithelial cells, alveolar epithelial cells, mucosal epithelial cells of the digestive system, renal glomerular epithelial cells, and hepatic parenchymal cells. Specific examples of endothelial cells include renal glomerular capillary cells, vascular endothelial cells, pulmonary arterial vascular endothelial cells, vascular endothelial cells of the placental vein, and aortic vascular endothelial cells. Specific examples of mesenchyme cells include fibroblasts, vascular smooth muscle cells, muscle cells, fat cells, glial cells, Schwann cells, and nerve cells. Specific examples of blood cells include macrophages.

Normal tissue cells and established cells to which primary culture technology can be applied are used in drug evaluation, but established cells are preferable. Particularly favorable cell strains serving as established cell lines of hepatic cells include FLC-1, FLC-2, FLC-3, FLC-4, FLC-5, FLC-6, FLC-7, HuH-6, HuH-7, C3A, HLE, HLF, HepG2, and so on.

Particularly favorable cells serving as established cell lines of intestinal cells include HT-29, IEC-6, Caco-2, and so on.

To trap the fine particles to which the cells adhere in a cell culture portion, the minimum width of the solution and fine particle inlet into the cell culture portion may be made larger than the maximum diameter of the fine particles, and the width of the outlet may be made smaller than the minimum diameter of the fine particles. In so doing, the fine particles to which the cells bond are dammed and do not flow beyond the cell culture portion. If necessary, measures may be taken to ensure that only cells desorbed from the fine particles, decomposition products thereof, or secretions thereof flow beyond the cell culture portion.

To measure the target cell product amount in the substance analysis portion, which is connected to the cell culture portion by a single passage, an antigen-antibody reaction, microchip electrophoresis, mass spectrometry, and so on may be used. Methods in which a target protein is labeled and specifically detected using fluorescence, an isotope, an enzyme, gold colloid, an oligohistidine tag, streptavidin, biotin, and so on are also effective.

To generate an antigen-antibody reaction in a substance analysis portion, an antibody which recognizes the cell product as an antigen may be fixed in advance in the passage of the substance analysis portion so that the cell product flowing from the cell culture portion can be trapped specifically. The antibody may be fixed directly onto the passage wall surface, or a carrier on which the antibody has been fixed in advance may be held in the passage.

A structure and a method for performing an antigen-antibody reaction such as those described in Japanese Patent Application Publication No. JP-A-2001-4628 may be cited as an example in which an antibody is fixed using a carrier. Specifically, an antibody which recognizes a cell product as an antigen is fixed on fine particles, and the antibody-fixing fine particles are dammed by making the minimum width of the solution and fine particle inlet into the substance analysis portion larger than the maximum diameter of the fine particles, and making the width of the passage interior or the outlet smaller than the minimum diameter of the fine particles. The cell product that flows from the cell culture portion may then be trapped specifically on the antibody fixed on the fine particles. To measure the amount of cell product trapped on the antibody, the antibody which recognizes the product as an antigen may be labeled in advance using an enzyme, a fluorescent substance, an isotope, a gold colloid, or the like, whereupon the labeled antibody may be led into the substance analysis portion. Then, the amount of the product trapped on the fine particles may be measured by introducing a matrix which is caused to color by an enzyme and measuring the amount of coloring, or by directly analyzing the fluorescent coloring or the photoabsorption of the gold colloid. By adjusting the amount of antibody or fine particles in the substance analysis portion at this time, setting can be performed to ensure that all of the products from the cells are trapped on the fine particles.

As regards the substrate material, a synthetic quartz substrate or a tempered glass substrate may be used, and a silicon substrate, a plastic having chemical resistance, and so on may also be used.

To form the passage, cell culture portion, substance analysis portion, and a through hole in the substrate, a micromachining technique based on a semiconductor manufacturing technique may be employed.

To prevent contamination, ethylene oxide gas sterilization or irradiation sterilization may be performed in the case of a molded resin such as plastic, and sterilization processing through autoclaving may be performed on a glass chip. The microchip may thereafter be handled in a non-contaminated environment such as a clean bench.

The fine particles introduced into the cell culture portion have a diameter of 1 mm or less, preferably between 10 and 300 μm, for example, and more preferably between 20 and 200 μm, for example, and may be selected appropriately according to the size of the cells to be evaluated. To make the cells easier to trap, the fine particles are preferably constituted by a material which enhances the adhesive strength of the cells.

Examples of the material for the fine particles include polystyrene, calcium (titanium) phosphate, alginic acid, poly (lactic acid-glycolic acid), chitosan, polyvinyl alcohol, calcium carbonate, hydroxyapatite, magnetic fine particles, cellulose, collagen, poly (methyl methacrylate), Sephadex, poly (vinyl-pyridine), dextran, and agarose.

Surface modification for accelerating cell adhesion may also be implemented on the fine particles, and examples of modification materials include collagen I, collagen III, collagen IV, collagen V, poly-L-lysine, poly-D-lysine, Matrigel, fibronectin, laminin, Cell Tak, vitronectin, and Attachin. Of these modification materials, that which is most suited to cultivation of the target cells should be used. For example, in the case of hepatic HepG2 cells, collagen I, collagen III, collagen IV, and collagen V are preferable.

To affix the cells to the fine particles described above, a fixed amount of the fine particles should be added to a cell suspension and then subjected to spinner culture between two hours to one night. Once the spinner culture is complete, the fine particles to which the cells are adhered may be filtered using filter paper or a film, whereby the cells which are not adhered to the fine particles can be removed. The number of cells affixed to the fine particles may be measured using a hemocytometer after dislodging the cells through trypsinization or the like. Alternatively, a fixed amount of a coloring reagent such as 3-(4,5-Dimethyl-2-thiazolyl)-2,5-diphenyl-2H tetrazolium bromide (MTT) or 2-(2-methoxy-4-nitrophenyl)-3-(4-nitrophenyl)-5-(2,4-disulfophenyl)-2H-tetrazolium, monosodium salt (WST-8) may be added to the cell-adhered fine particle solution for one to three hours at 37° C., and the cell count can be measured by measuring the absorbance of the generated formazone dye.

Similarly to a coloring method, a fluorescent dye such as 2′, 7′-bis-(carboxyethyl)-5-(and-6)-carboxyfluorescein) or Alamar Blue may be added to the fine particle solution, and the cell count may be measured by measuring the fluorescence intensity.

To fix the antibody on the passage wall surface or fine particles, a physical or chemical method may be employed. An example of a physical method is to place an antibody solution in the passage or around the fine particles and leave it for two to three hours at 37° C. or overnight at 4° C. An example of a chemical method is to introduce a functional group such as a carboxyl group or an amino group into the passage or onto the surface of the fine particles and fix the antibody through covalent bonding.

An embodiment of the present invention will be described below.

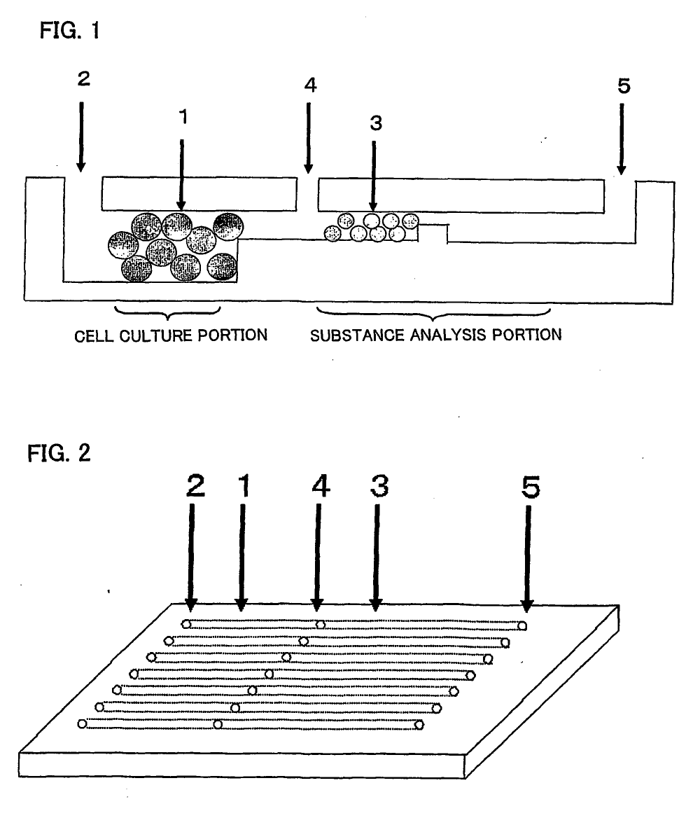

First, the structure of the microchip according to the present invention can be described using the example shown in FIG. 1, for example. As shown in FIG. 1, the microchip of the present invention comprises the aforementioned cell culture portion, which comprises fine particles (1) having a diameter of no more than 1 mm and having cells adhered thereto, a fine particle and solution introduction portion (2) having a minimum width which is larger than the diameter of the fine particles (1), and a dividing portion having a width which is smaller than the diameter of the fine particles (1), and an introduction portion (4) for introducing fine particles (3) having a fixed antibody for trapping an antigen which is secreted from the cells, and a labeled antibody or an antigen-antibody reaction reagent, separately to the aforementioned substance analysis portion.

Further, in the example in FIG. 1, a drain portion (5) is provided at an end portion of the substance analysis portion.

The fine particles (3) having the fixed antibody serve as a reaction solid phase for an immune antigen-antibody reaction, and glass beads or polymer beads made of polystyrene or the like may be employed as these fine particles (3), for example. In the microchip of this invention, the fine particles (3) have a diameter of no more than 1 mm, for example between 15 and 85 μm, and preferably between 40 and 65 μm.

To introduce the solution or fine particles into the introduction portions (2) and (4), a pump or syringe pump used in analysis chromatography may be employed. Further, by providing a check valve system prior to introduction into the passage, it is possible to introduce various reagents and samples selectively. As a result, the introduction of each solution, reagent, sample, and fine particles, and the accompanying reactions, can all be automated by machine.

As regards photometric analysis of the coloring obtained as a result of the antigen-antibody reaction, analysis using a thermal lens microscope, such as that described in Japanese Patent Application Publication JP-A-2001-4628, for example, may be cited as a representative example thereof. This analysis may be performed directly on the fine particles (3) to which the reaction products are adhered or on the passage from the substance analysis portion onward. Instead of analysis using a thermal lens microscope, the coloring may be measured by means of fluorescence analysis, chemiluminescence analysis, and so on.

FIG. 2 shows an example of a case in which a large number of samples is measured.

As shown in FIG. 2, in the microchip of the present invention, one or a plurality of the passage structures described above may be formed in a single substrate, or one or a plurality of substance analysis portions may be connected to a single cell culture portion. Further, the passage outlet of a microchip comprising a cell culture portion and the passage inlet of a microchip comprising a substance analysis portion may be connected by an external tube or the like such that a series of measurement can be performed. In this case, individual temperature adjustment of the cell culture portion and substance analysis portion can be performed easily.

In all cases, cell response to a drug can be evaluated easily and in a short reaction time by means of the microchip of the present invention.

The following method may be cited as an example of a method for evaluating cell responses.

The fine particles (1) to which the cells are affixed in advance and the fine particles (3) on which an antibody to the evaluation subject cell product is fixed are introduced into the cell culture portion and the analysis portion, respectively. Next, a culture medium containing an added drug is introduced continuously through the introduction portion (2) and the generated culture solution is introduced as is into the fine particles (3), whereby the target cell product in the culture solution is trapped on the fine particles (3). Next, a labeled antibody and a coloring reagent are introduced through the inflow portion (4) to induce a reaction in the antibody on the fine particles (3), whereupon the generated solution is led as is into the substance analysis portion and subjected to coloring analysis using a thermal lens microscope or the like.

An appropriate representative example of a labeled antibody when using a coloring reagent is horseradish peroxisome (HRP). In certain cases, gold colloid or a fluorescent labeled antibody may be used, but in this case, the fine particles (3) are preferably analyzed directly.

After measuring the antigen-antibody reaction, the coloring reagent to be used to measure the cell count is introduced through the introduction portion (2), and the coloring of the solution that flows out from the introduction portion (4) is detected by an absorptiometer or the like. In so doing, the number of cells in the cell culture portion can be measured.

According to the present invention as described above, cell cultivation, cell secretion measurement, and cell count measurement can be performed simply, in a short amount of time, and with a high degree of precision.

### EXAMPLES

The present invention will be described in further detail below using examples. However, the present invention is not limited to these examples.

### Example 1

### Assay of Apolipoprotein Secreted by Hepatic Cells

Using the microchip of the present invention, the drug-induced secretion of ApoA1 from human hepatoma HepG2 cells was measured. The microchip used in the experiment had a microchannel passage with a depth of 100 μm and a width of 200 μm. In the microchip, a cell culture portion had an introduction portion with a depth of 300 μm and an outlet with a depth of 100 μm and a width of 200 μm, and a microchannel dividing portion having a depth of 15 μm was created in a tempered glass substrate of the substance analysis portion so that fine particles could be trapped.

### (1) Cells and Culture

The HepG2 cells were suspended in a Dulbecco's Modified Eagle's Medium (DMEM) low glucose culture medium containing 10% fetal calf serum (FCS), added to a 20 mL beaker at a concentration of 2×10⁶ cells/mL (10 ml) together with a 0.4 g/mL concentration collagen-coated microcarrier (manufactured by SOLOHILL ENGINEERING, INC., average diameter 108 μm, “carrier” hereafter), and subjected to spinner culture overnight at 37° C. Following cultivation, the carrier was filtered using a cell strainer (70 μm nylon mesh, manufactured by BD Falcon), whereupon the carrier was washed in the culture medium and re-suspended. Further, a part of the carrier suspension affixed with the cells was dispensed, and using WST-8 reagent (manufactured by Kishida Chemical Co.), the cell count was measured in accordance with the attached instruction manual. More specifically, 10 μL of a 5 mM WST-8 reagent solution was added to 100 μL of an appropriately diluted solution of the carrier suspension, and the mixture was heated at 37° C. for two hours. The 450 nm absorbance of the generated formazan dye was then measured using a 96 well microplate reader (manufactured by BIORAD). Following measurement of the cell count, the carrier suspension (approximately 2000 units/passage) was injected into the cell culture portion of the microchip described above to realize a cell count of 1×10⁴ cells/passage, whereupon the aforementioned culture medium was introduced over one hour at 37° C. and at a flow velocity of 0.5 μL/minute using a microsyringe pump to remove the cells not adhered to the fine particles from the cell culture portion.

### (2) Drug Addition and ApoA1-ELISA

An anti-human ApoA1 monoclonal antibody HDL110 (manufactured by MABTECH) was added to a solution of 25 μm diameter polystyrene beads (manufactured by Funakoshi)/PBS (phosphate buffered saline), and left for 18 hours at 4° C. Following washing in the PBS, 1% BSA (bovine serum albumin) was added, and the mixture was left for 1.5 hours at room temperature. The antibody-bound beads were washed in PBS containing 0.05% Tween 20 (PBS-T), and then re-suspended in the PBS-T. The antibody beads solution was then injected (at approximately 10,000 units/passage) into the substance analysis portion of the microchip following the completion of cell cultivation therein, whereupon a solution of a drug known to have an ApoA1 secretomotor effect (the compound numbered 27 in the example described in Japanese Patent Application Publication JP-A-2002-053557 (abbreviated to Compound 1 hereafter)), that has been diluted in an appropriate culture medium, was introduced over 17 hours at 37° C. and at a flow velocity of 0.5 μL/minute. At this time, the solution introduced into the cell culture liquid was discharged entirely through the antibody beads in the substance analysis portion. Following introduction, a 0.5 mM WST-8 reagent solution was introduced over one hour at 37° C. and at a flow velocity of 0.5 μL/minute, whereupon the 450 nm absorbance of the generated formazan dye was measured using a 96 well microplate reader (manufactured by BIORAD), and the cell count per passage was measured. In the substance analysis portion, the antibody beads were washed in PBS-T, whereupon a biotin-labeled anti-human ApoA1 monoclonal antibody HDL44 (manufactured by MABTECH) was introduced into the aforementioned culture medium over ten minutes at a flow velocity of 1 μL/minute and at 37° C., and washed in PBS-T. Further, streptavidin horseradish peroxidase (HRP) was introduced under the same conditions as above and washed in the PBS-T. Next, an ABTS solution (manufactured by Sigma) was introduced as a coloring matrix at a flow speed of 1 μL/minute, and the generated coloring was measured with a thermal lens microscope using a method such as that described in Japanese Patent Application Publication JP-A-2000-356611 and Japanese Patent Application Publication JP-A-2001-4628. More specifically, the passage 1 cm downstream of the antibody beads was irradiated with excitation light at 633 nm, and coloring was detected using a 488 nm probe beam as a thermal lens signal. Note that purified human ApoA1 (ALP10; manufactured by Chemicon International) diluted in PBS-T was used for the standard curve.

### (3) Evaluation of Response to Drug

The quantity of ApoA1 secreted into the culture medium by the HepG2 cells was corrected in accordance with the cell count (450 nm absorbance), whereupon the variation relative to a control (% of control) was calculated, and the ApoA1 production promotion action of the aforementioned drug was evaluated. The results are shown in Table 1.

### Table 1

### Compound 1

### Variation

As shown in Table 1, it was found that when the microchip of the present invention is used, ApoA1 secretion promotion based on drug stimulation of the cells can be evaluated effectively.

### Example 2

### Assay of Apolipoprotein Secreted by Enteric Cells

Using the microchip of the present invention, the secretion of ApoB from human colon cancer Caco-2 cells was measured. Two types of microchip, namely a cell cultivation chip and a substance analysis chip, were used in the experiment, each having a microchannel passage with a depth of 100 μm and a width of 200 μm. The cell cultivation chip had a cell culture portion including an introduction portion with a depth of 300 μm, an outlet with a depth of 100 μm and a width of 200 μm. The substance analysis chip had a microchannel dividing portion with a depth of 15 μm, which was created in a tempered glass substrate as substance analysis portion, and structured so that fine particles could be trapped.

### (1) Cells and Culture

The Caco-2 cells were suspended in an RPMI-1640 culture medium (manufactured by Kohjin Bio) containing 10% fetal bovine serum (FBS), added to a 20 mL beaker at a concentration of 6×10⁶ cells/mL (10 ml) together with a 0.4 g/mL concentration collagen-coated microcarrier (manufactured by SOLOHILL ENGINEERING, INC., average diameter 108 μm, “carrier” hereafter), and subjected to spinner culture for one week at 37° C. Following cultivation, the carrier suspension (approximately 2000 units/passage) was injected into the cell culture portion of the aforementioned cell cultivation chip, whereupon the aforementioned culture medium was introduced at 37° C. and at a flow velocity of 0.2 μL/minute using a microsyringe pump. At this time, the carrier not affixed with cells was injected into a separate cell culture portion as a cell-free control, and the culture medium was introduced thereto in a similar fashion.

### (2) ApoB-ELISA

An anti-human ApoB monoclonal antibody 2-B4 (manufactured by Intracell) was added to a solution of 25 μm diameter polystyrene beads (manufactured by Funakoshi)/PBS (phosphate buffered saline), and left for 18 hours at 4° C. Following washing in the PBS, 1% BSA (bovine serum albumin) was added, and the mixture was left for 1.5 hours at room temperature. The antibody-bound beads were washed in PBS containing 0.05% Tween 20 (PBS-T), and then re-suspended in the PBS-T. To measure the ApoB production quantity, the antibody beads solution was injected (at approximately 10,000 units/passage) into the substance analysis portion of the substance analysis chip, whereupon the passage outlet of the cell culture chip and the inlet of the substance analysis chip were connected by a capillary tube, and an RPMI-1640 culture medium not containing FBS was introduced from the cell cultivation chip over 17 hours, at 37° C., and at a flow velocity of 0.5 μL/minute. At this time, the solution introduced into the cell culture liquid was discharged entirely through the antibody beads in the passage of the substance analysis chip. Following introduction, the antibody beads were washed in PBS-T in the substance analysis portion, whereupon a horseradish peroxidase (HRP)-labeled anti-human ApoB polyclonal antibody (manufactured by The Binding Site) was introduced over ten minutes at a flow velocity of 5 μL/minute and at room temperature, and washed in PBS-T. Next, a TMB solution (manufactured by Sigma) was introduced as a coloring matrix over 20 minutes at a flow speed of 5 μL/minute and at room temperature, and the absorbance (650 nm) of 100 μL of the solution was measured using a 96 well microplate reader (manufactured by Nihon Molecular Devices Corporation).

### (3) Evaluation of ApoB Production Over Time

The quantity of ApoB secreted into the culture medium by the Caco-2 cells adhered to the carrier was calculated from the relative absorbance intensity (% of control) to the cell-free control, and the amount of produced ApoB per cultivation time was evaluated. The results are shown in Table 2.

TABLE 2

Cell culture liquid
Cultivation time (days)
Variation

As shown in Table 2, it was found that when the microchip of the present invention is used, the amount of ApoB produced through secretion from the Caco-2 cells per cultivation time can be evaluated effectively.

### INDUSTRIAL APPLICABILITY

The microchip of the present invention is capable of performing cell cultivation and analyzing a substance secreted by the cells continuously, and is therefore useful as a tool for evaluating cell functions and responses to drugs.

1. A microchip for evaluating a cell response, comprising a cell culture portion which is capable of holding a fine particle to which a cell is adhered.

2. The microchip according to claim 1, comprising a passage formed in the interior of the microchip so as to be capable of carrying a liquid, a liquid inlet and outlet connected to two end portions of the passage, and a cell culture portion provided in the interior of the passage, which is capable of holding fine particles with a diameter of no more than 1 mm.

3. The microchip according to claim 1, comprising a substance analysis portion provided in a latter half of the passage in relation to the cell culture portion for measuring a secretion from the cell.

4. A microchip, wherein the passage outlet of the microchip according to claim 1 and a passage inlet of a microchip having a substance analysis portion are connected by an external tube.

5. A method of measuring a cell secretion using the microchip according to claim 3.

6. A method of measuring a cell secretion by performing an enzyme immunoassay in the substance analysis portion according to claim 3.

7. The measuring method according to claim 5, wherein measurement is performed while causing a liquid to flow along the passage of the microchip continuously.

8. The measuring method according to claim 5, wherein the cell secretion is apolipoprotein A1.

9. The measuring method according to claim 5, wherein the cell secretion is apolipoprotein B.

10. A screening method for a drug which is active in varying an amount of secretion from a cell, using the measuring method according to claim 5.

11. The drug screening method according to claim 10, wherein the cell secretion is apolipoprotein A1.

12. The drug screening method according to claim 10, wherein the cell secretion is apolipoprotein B.

13. The drug screening method according to claim 11, wherein the cell is a HepG2 cell.

14. The drug screening method according to claim 12, wherein the cell is a Caco-2 cell.
